(19) United States
(12) Patent Application Publication
(10) Pub. No.: US 2005/0090747 A1
(43) Pub. Date: Apr. 28, 2005

(54) Automatic alias avoidance for doppler audio

(76) Inventor: David W. Clark, Windham, NH (US)

Correspondence Address: PHILIPS INTELLECTUAL PROPERTY & STANDARDS, P.O. BOX 3001, BRIARCLIFF MANOR, NY, 10510, US

(21) Appl. No.: 10/957,156

(22) Filed: Oct. 1, 2004

Related U.S. Application Data
(60) Provisional application No. 60/509,010, filed on Oct. 6, 2003.

(51) Int. Cl. A61B 8/02

(52) U.S. Cl. 600/453

(57) ABSTRACT

The instantaneous peak positive and negative frequencies of a Doppler spectral image are determined using the spectral column signal mask derived from the instantaneous peak frequency function of the ultrasound system. Using the determined peak frequencies, the audio wraparound frequency is adjusted so as to substantially avoid aliasing effects.

### CROSS REFERENCE TO RELATED CASES

Applicant claims the benefit of Provisional Application Ser. No. 60/509,010, filed Oct. 6, 2003.

### BACKGROUND OF THE INVENTION

This invention relates to ultrasonic imaging systems and, in particular, to the audio output of an ultrasonic imaging system in spectral Doppler operational mode.

Ultrasonic medical transducers are used to observe the internal organs of a patient. The ultrasonic range is described essentially by its lower limit: 20 kHz, roughly the highest frequency a human can hear. The medical transducers emit ultrasonic pulses which, if not absorbed, echo (i.e., reflect), refract, or are scattered by structures in the body. Most of the received signal is from scattering, which is caused by many small inhomogeneities (much smaller than a wavelength) making a small part of the wave energy disperse in all directions. The signals are received by the transducer and these received signals are translated into images. The sum of the many scattered waves of random phase cause the resulting image of the received signals to be speckly.

There are a number of imaging and/or diagnostic modes in which an ultrasonic system operates. The most fundamental modes are A Mode, B Mode, M Mode, and 2D Mode. The A Mode is amplitude mode, where signals are displayed as spikes that are dependent on the amplitude of the returning sound energy. The B Mode is brightness mode, where the signals are displayed as various points whose brightness depends on the amplitude of the returning sound energy. The M Mode is motion mode, where B Mode is applied and a strip chart recorder allows visualization of the structures as a function of depth and time.

The 2D Mode is the fundamental two-dimensional imaging mode. In 2D mode, an ultrasonic transmission beam is swept back and forth so that internal structures can be seen as a function of depth and width. By rapidly steering the beams from left to right, 1 2D cross-sectional image may be formed. There are other imaging modes, which also image in two dimensions (and also in three dimensions), and these are often referred to by their own names, usually based on the type of technology/methodology (such as “harmonic” or “Doppler”) used to produce the image.

Several modes of imaging are dependent on the Doppler effect, the phenomena whereby the frequency of sound from an approaching object has a higher frequency and, conversely, sound from a receding object has a lower frequency. In ultrasonic systems, this effect is used to determine the velocity and direction of blood flow in a subject. Continuous wave (CW) Doppler mode transmits a continuous ultrasound signal and determines the frequency shift of the scattering echo received from moving targets, e.g., blood cells. By contrast, pulsed Doppler mode transmits a periodic pulse of ultrasound energy and determines the phase or time shift of the received series of pulse echoes, not on the frequency shift of a single echo. Major Doppler imaging techniques include color flow Doppler, spectral Doppler, and power Doppler.

In color flow imaging (CFI), sample volumes are detected and displayed utilizing color mapping for direction and velocity flow data. Most commonly, this results in a grey-scale image with superimposed colors indicating blood-flow velocity and direction. Color mapping formats include BART (Blue Away, Red Towards), RABT (Red Away, Blue Towards), or enhanced/variance flow maps where color saturations indicate turbulence/acceleration and color intensities indicate higher velocities. Some maps use a third color, green, to indicate accelerating velocities and turbulence.

Power Doppler does not show the direction of flow, but rather the colors in a power Doppler image indicate whether any flow is present. The Doppler signals are processed differently in power Doppler imaging: instead of estimating mean frequency and variance through autocorrelation, the integral of the power spectrum is estimated and color-coded. Because power Doppler imaging is based on the total power of the received Doppler signal, the results are independent from the velocity of the blood-flow.

Spectral Doppler refers to ultrasound methods, whether pulsed or CW Doppler, which present the results of flow velocity measurements as a “spectral display”. A spectral display shows the entire Doppler frequency shift (or blood-flow velocity) range present in the measurements. Spectral Doppler usually also includes stereo audio output of the flow signal. An “amplitude vs. frequency spectral display” shows the amplitudes of all the Doppler frequency shifts present at a particular moment in time. The more common “time-velocity spectral display” shows how the full spectrum of Doppler frequency shifts (or blood-flow velocities) varies over time. FIG. 1 shows a time-velocity spectral display of a carotid artery. As can be seen in FIG. 1, the abscissa of the time-velocity spectral display represents time while the height represents speed (in cm/s).

Spectral Doppler techniques typically employ two types of signal output: visual output in the form of a spectrogram, such as the one in FIG. 1, and audio output through stereo speakers.

Regardless of the mode of operation (whether pulsed or continuous wave), the sampling process used in Doppler imaging introduces a problematic artifact known as “aliasing” which will be explained in reference to FIGS. 2A and 2B. FIG. 2A represents the received signal 200 in the frequency domain (i.e., the graph shows the received signal in terms of its frequency content) before sampling. At the center of the graph is a horizontal line labeled “0” and “DC” which stand for zero frequency and, equivalently, direct current, respectively. The positive frequencies are above the DC line (thus representing motion toward the ultrasound transducer), and the negative frequencies are below the DC line (thus representing motion away from the ultrasound transducer). Two horizontal lines 210 and 215 placed equidistantly above and below the DC line represent the positive and negative cutoff frequencies, respectively, of the clutter filter (a high-pass filter which must be used to reduce or eliminate unwanted high-amplitude, low-velocity signals from the in-coming signals).

In an ultrasound system, the incoming signal is “sampled” before going through spectral analysis, and the sampling rate Fₛ is equivalent to the Pulse Repetition Frequency (PRF). According to the Nyquist Sampling Theorem, any frequency in a signal being sampled that is greater than half the sampling rate Fₛ (i.e., Fₛ/2) will cause aliasing. This is shown in FIG. 2B, where we have the received signal 200 from FIG. 2A after having been sampled at sampling rate Fₛ. The portion labeled 30A of signal 200 that was above the Fₛ/2 line has been aliased, and now appears as a negative frequency in the bottom of the graph. Aliasing can result in either direction; in other words, negative frequency content below −Fₛ/2 would wrap around to the positive side of the sampled signal. Without correction, the aliasing shown in FIGS. 2A and 2B (the positive frequency content 30A of signal 200 appearing as negative frequency content in the sampled signal) would result in blood flow towards the ultrasonic transducer being mapped as blood flow away from the ultrasonic transducer.

In prior art Doppler imaging systems, the operator or the ultrasound system itself can compensate for aliasing by inverting or shifting the frequency content of the sampled signal. Inverting the signal, which results in the visual display or spectrogram being inverted, provides better intuitive visualization for the operator. If the total spectral bandwidth of the signal (the width of signal 30 from top to bottom in FIG. 2A) is less than the PRF, the baseline can be shifted so that the signal unwraps. To be more exact, it is the positive and negative wrap frequencies that change so as to move the “window” of data (which is normally from −PRF/2 to +PRF/2) up or down to encompass the entire signal. If the spectral bandwidth is more than, or close to, the PRF (the width of the “window”), the PRF itself will need to be increased. For example, the negative peak frequency in FIG. 2B is very close to −PRF/2, thus, if the “window” was moved upwards (in order to put portion 30A back up on the positive side), the bottom wrap frequency of the “window” would also move up, causing the negative peak frequencies to begin aliasing.

However, in most prior art systems, it is only the video signal that is “anti-aliased”, thus leaving the audio signal untouched, or the audio signal receives the same anti-aliasing processing as the video signal. If the audio signal does not undergo some anti-aliasing technique, the audio signal is no longer an accurate reproduction of the signal and no longer is consonant with the spectral display. It is standard practice for the stereo audio output to consist of a right channel bearing the positive frequency information (above DC) and the left channel bearing the negative frequency information (below DC). Thus, when the audio signal is aliased, it jumps from one speaker to the other, without changing audio frequency as heard by the user. Furthermore, if the frequency of the true audio signal increases further beyond Fₛ/2 (i.e., PRF/2), the audio signal which is already coming from the wrong speaker will decrease in frequency rather than increase.

On the other hand, if the audio signal is adjusted in the same manner as the video output, there should be no aliasing most of the time. Two examples of ultrasound systems in which the audio output is adjusted in a similar manner to the video output are U.S. Pat. No. 5,676,148 to Koo et al. (which patent shall be hereinafter referred to as the “Koo patent” and the system described therein as the “Koo system”) and U.S. Pat. No. 5,553,621 to Otterson (which patent shall be hereafter referred to as the “Otterson patent” and the system described therein as the “Otterson system”), both of which are hereby incorporated by reference in their entirety. As shown in FIG. 3 (which is a reproduction of FIG. 1 of the Koo patent), the echo signals 14 return from the sample volume (in this case, blood vessel 13) and are converted into electrical signals, which are processed by the various modules in box 16 before being sampled by sampler 18. The output of sampler 18 is input into both display processor 208 which outputs to display 20A and audio splitter 21A which outputs to stereo speakers 26.

Both the Koo and the Otterson systems add a zero inserter 22, as can be seen in FIG. 4 (which is a reproduction of FIG. 5 of the Koo patent), which inserts zeros into the data 19 received from the sampler 18 before that data enters into final processing 24 to be output to speakers 26. The insertion of zeros between sample points in the incoming signal effectively doubles the sampling rate Fₛ. The zero-inserted sample data is then filtered by a filter having an alterable cutoff frequency corresponding to the spectral adjustment of the video output.

As was stated above, if the audio signal is adjusted in the same manner as the video output, there should be no aliasing most of the time. But aliasing can still occur due to misadjustment or a rapid change in signal. In the case of aliasing in which the frequency jumps from a low frequency to a high frequency, it produces a very irritating screech when it wraps. Furthermore, if the audio signal has the same baseline offset as the video signal, the background noise amplitude and pitch in the two stereo channels are different, because one of the channels has more bandwidth than the other, and background noise power is generally proportional to bandwidth.

Therefore, there is a need for an technique that automatically avoids audio aliasing where possible, regardless of how the spectral baseline offset is adjusted; which degrades gracefully without irritating screeches when aliasing is unavoidable or ambiguous; and which makes any difference of background noise between the two stereo channels imperceptible.

### SUMMARY OF THE INVENTION

One objective of the present invention is to provide a method and system for automatically avoiding audio aliasing when possible, regardless of how the spectral baseline offset for the video is adjusted.

Another objective of the present invention is to provide a method and system for automatically avoiding audio aliasing when possible, and whose audio output degrades substantially smoothly (e.g., without irritating screeches) when aliasing is unavoidable or ambiguous.

Yet another objective of the present invention is to provide a method and system for automatically avoiding audio aliasing when possible, and for making any difference of background noise between the two stereo channels imperceptible.

These and other objectives are met by the present invention, in which the signal mask derived from the instantaneous peak frequency function of the ultrasound system is used to determine the peak positive and negative frequencies of the Doppler spectrum. In the presently preferred embodiments, the signal mask is used to perform at least one of tracking the peak frequencies and determining the instantaneous peak frequencies, and then the audio wraparound frequency is adjusted so as to substantially avoid aliasing effects.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 is a time-velocity spectral display showing how the full spectrum of Doppler frequency shifts (or blood-flow velocities) varies over time in a carotid artery according to conventional spectral Doppler imaging;

FIG. 2A is a graph showing received spectral Doppler data;

FIG. 2B is a graph showing the spectral Doppler data 200 of FIG. 2A after being sampled;

FIG. 3 is a block diagram showing the components of a prior art ultrasound system for producing spectral Doppler video and audio output, where the audio output is adjusted in the same manner as the video output in order to avoid aliasing effects;

FIG. 4 is a block diagram showing the components of another prior art ultrasound system for producing spectral Doppler video and audio output, where the audio output is adjusted with a zero inserter before processing in order to avoid aliasing effects;

FIG. 5A is a schematic representation of the signal data array of FIG. 2B being rearranged into a more convenient form;

FIG. 5B is a schematic representation of a spectral column in the rearranged signal data array of FIG. 5A being;

FIG. 6A is a flowchart of an exemplary method for avoiding aliasing effects according to one presently preferred embodiment of the present invention;

FIG. 6B is a flowchart of an exemplary method for avoiding aliasing effects according to another presently preferred embodiment of the present invention; and

FIG. 6C is a flowchart of an exemplary method for avoiding aliasing effects according to yet another presently preferred embodiment of the present invention.

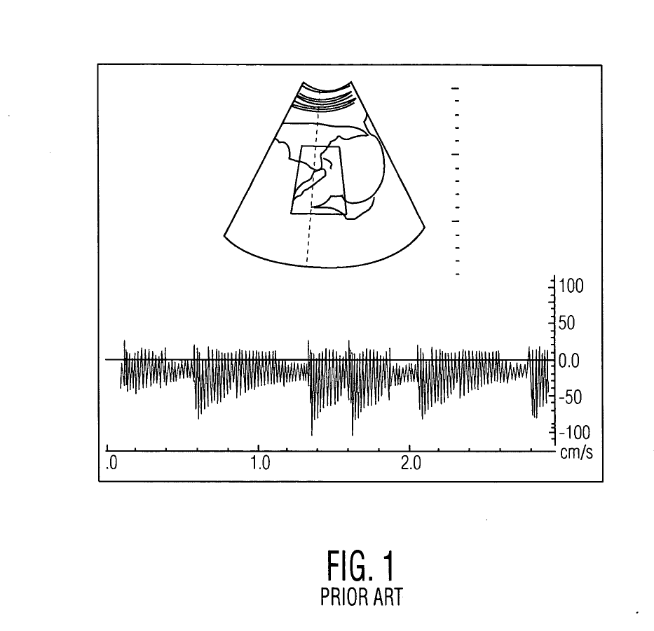

### DESCRIPTION OF THE PREFERRED EMBODIMENTS

In general, the present invention is directed to a system and method for ameliorating the effects of aliasing on the audio output of an ultrasonic imaging system performing spectral Doppler imaging. In the presently preferred embodiments, the audio wrap frequency is changed based on the binary signal mask used by the ultrasound system to determine the instantaneous peak frequency of a spectral column within the spectral image. This same alias avoidance technique may also be applied to the spectral video display or to derived waveforms and/or measurements.

Because the peak frequency, which corresponds to the peak velocity of blood flow, is important in the clinical diagnosis of such conditions as arterial stenosis or valvular regurgitation, present ultrasound systems typically have the ability to determine the instantaneous peak frequency (on both positive and negative sides) in a spectral column of the Doppler spectrogram image. The determination of the instantaneous peak frequency usually involves some combination of smoothing and amplitude thresholding to segment the instantaneous spectrum, i.e., one spectral column in the spectrogram, into signal regions and noise regions. In the resulting “signal mask”, the boundaries of the signal regions which are farthest away from zero frequency (DC) are the instantaneous peak frequencies (one for each side of the spectrum: negative and positive). Typically, the peak frequency determination function in present ultrasound systems does not attempt to unwrap aliasing; thus, the automatically determined peak frequencies do not extend beyond the displayed frequency range.

In one presently preferred embodiment of the present invention, the spectral column signal mask is used both to determine the present instantaneous peak frequencies and to track the peak frequency from one spectral column to the next. Thus, in this presently preferred embodiment, the search for the new peak frequency on the new signal mask starts at the previously found peak frequency, proceeding either toward or away from DC depending on whether the mask at the present frequency indicates the presence or absence of a signal.

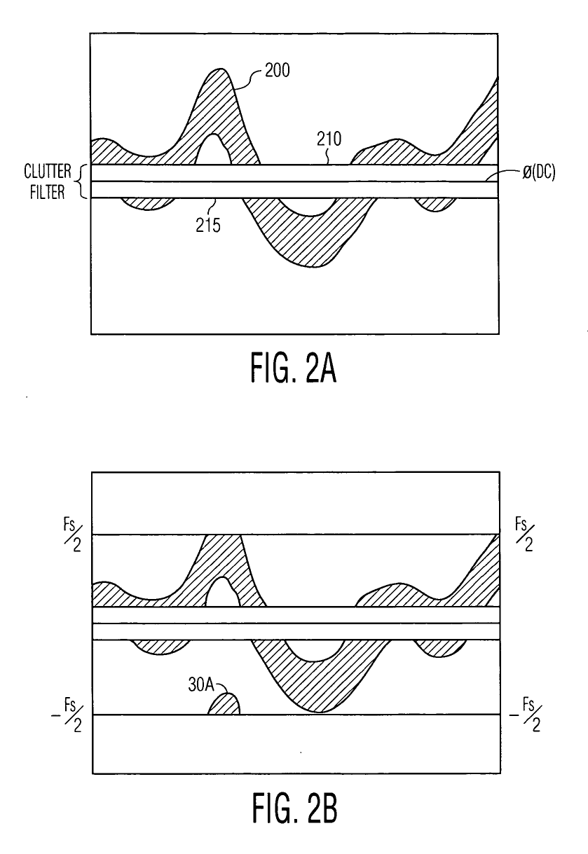

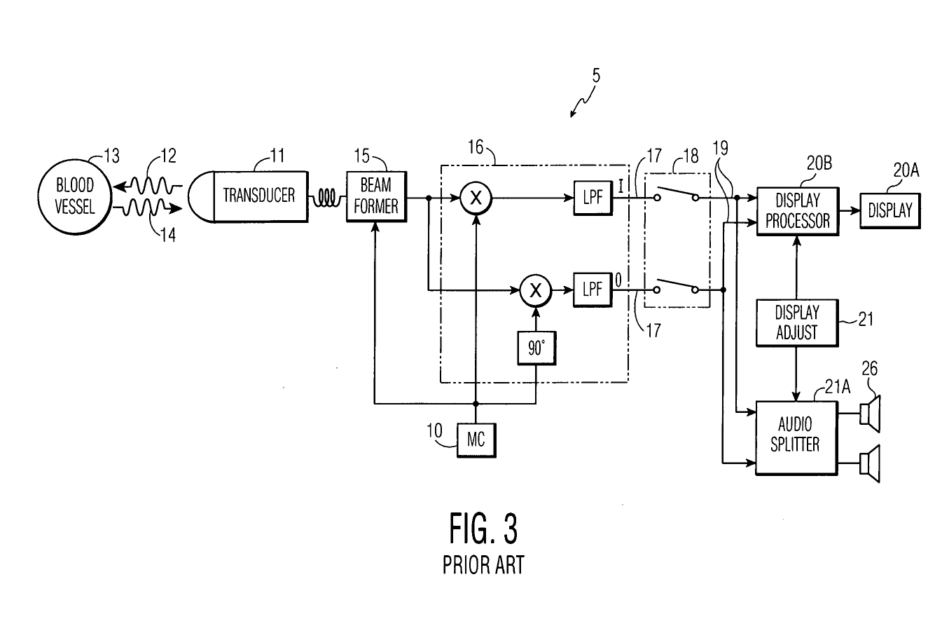

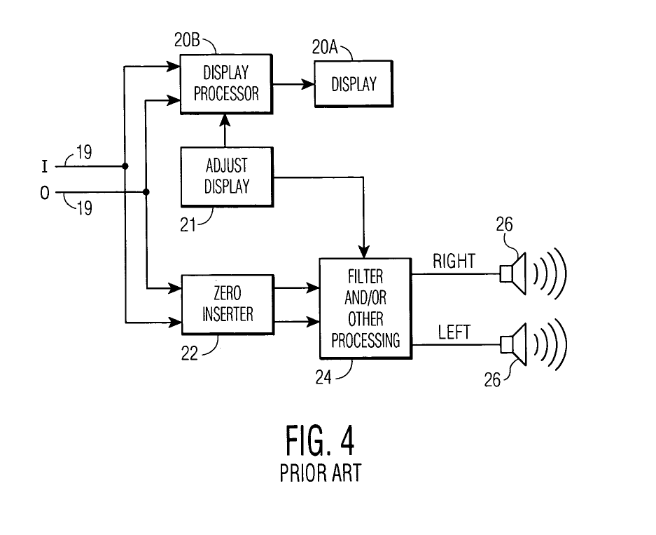

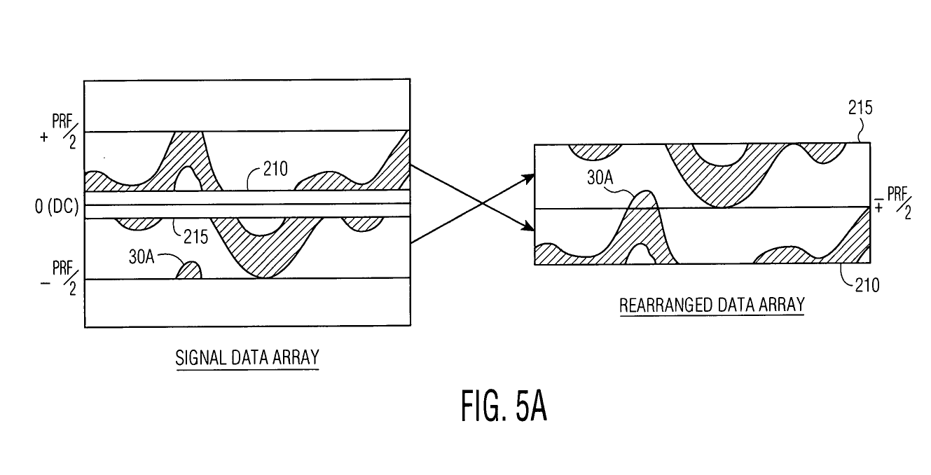

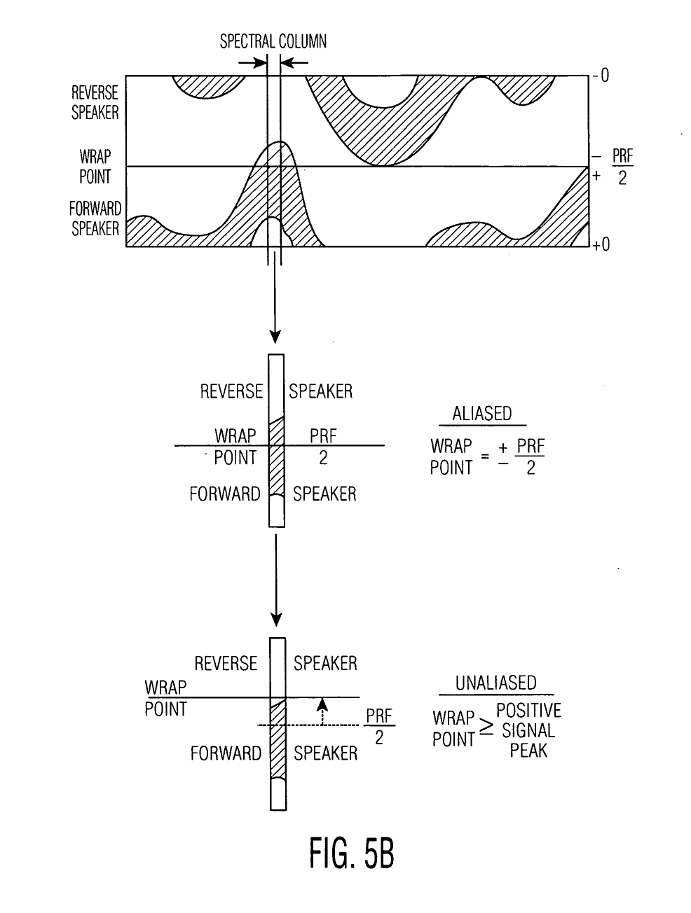

Ultrasound systems typically re-arrange the signal data array in memory in order to simplify the performance of various algorithms. Such a rearrangement is shown in FIG. 5A, where the graph on the right side is the signal data array of FIG. 2B, and the graph on the left side shows the rearranged data array. In the rearranged data array, the negative spectrum is set above the positive spectrum, thus making the line between them equal to PRF/2, the top line equal to the negative cutoff frequency 215 of the clutter filter, and the bottom line equal to the positive cutoff frequency 210 of the clutter filter. Once the signal data array is arranged in this manner, the new peak frequency is unambiguous, as long as the previous peak frequency was unambiguous (if the positive and negative signals do not overlap), because the aliased spectrum flow is unfolded in such an arrangement. For example, as shown in the left-hand graph of FIG. 5A, once rearranged, the aliased portion 30A of the signal appears as a continuation of the rest of its spectrum.

According to one presently preferred embodiment of the present invention, the default audio wraparound frequency is PRF/2, when neither positive nor negative peak signal frequency exceeds PRF/2. That ensures that the background noise is balanced between the stereo channels. However, when either the negative or positive peak frequency exceeds PRF/2, the audio wraparound frequency is set equal to it (or near it) so that the audio signal is not aliased. This unbalances the background noise, but only when there is a relatively high frequency signal, so that the noise unbalance is not easily perceptible.

A graphic example of the application of the method according to the presently preferred embodiment is shown in FIG. 5B. At the top of FIG. 5B, the rearranged spectral data array of FIG. 5A is shown with a spectral column (i.e., an instantaneous signal mask) highlighted within it. In this top graph, the wraparound frequency (or wrap point) is equal to PRF/2; thus, the speaker playing the forward channel would be producing the equivalent of the bottom half of the top graph (from +0 to +PRF/2), and the speaker playing the reverse channel would be producing the equivalent of the top half of the top graph (from −PRF/2 to −0). In the middle of FIG. 5B, the highlighted spectral column is shown alone, with the bandwidth being played by the reverse speaker and the bandwidth being played by the forward speaker being separated at the wrap point, which is equivalent to PRF/2. Because the wrap point equals PRF/2, the aliased portion of the positive frequency signal will play on the reverse speaker instead of the forward speaker. In the spectral column on the bottom of FIG. 5B, the wrap point has been moved so that it exceeds the peak positive frequency in the spectral column; thus, the entire positive signal region will be played on the forward speaker and aliasing will be avoided.

There may be situations where the positive and negative peak frequencies can not be resolved, for example, when positive and negative signal spectra overlap. One possible solution is to create a new signal mask using a higher threshold to try to separate the signals. However, if continual efforts to find the peak frequencies are unsuccessful, the system according to the presently preferred embodiment of the present invention sets the audio wrap frequency to PRF/2. Such a default audio wrap frequency ensures that the unavoidable aliasing is at least not too irritating, because the signal does not change frequency when it wraps and the stereo channels sound balanced.

At the beginning of the processing, or if the previous peak frequencies could not be resolved, the search for current peak frequencies can start at either PRF/2 (the middle of the rearranged mask array), or at the baseline-offset wrap frequency of the spectral display. Two potentially difficult situations are:

(1) Venous flow with a low PRF, such that the peak signal frequency is beyond the search start frequency for most or all of the cardiac cycle, but the signal is very broadband; and

(2) Arterial flow in a large vessel (e.g., carotid), such that a relatively narrowband signal is entirely beyond the search start frequency during peak systole. A variety of heuristic rules can be used to ensure that the peak frequency tracking resolves correctly as rapidly as possible in the above-listed situations. The following are several alternative rules to correctly associate a signal region in the mask array with the positive or negative side of the spectrum in no more than a fraction of a second:

(a) If the signal region ever closely approaches the edge of the clutter filter stopband, then assign it to that side of the spectrum; or

(b) Assign the signal region to the side of the spectrum that it covers to the greater extent, when averaged over time.

Other possible rules for ensuring correct startup or correction of tracking errors will be apparent to those skilled in the art, and are included in the scope of the invention. In any case, once the signal region is assigned to the correct spectrum side, frequency tracking proceeds unambiguously as long as the positive and negative signal spectra do not overlap.

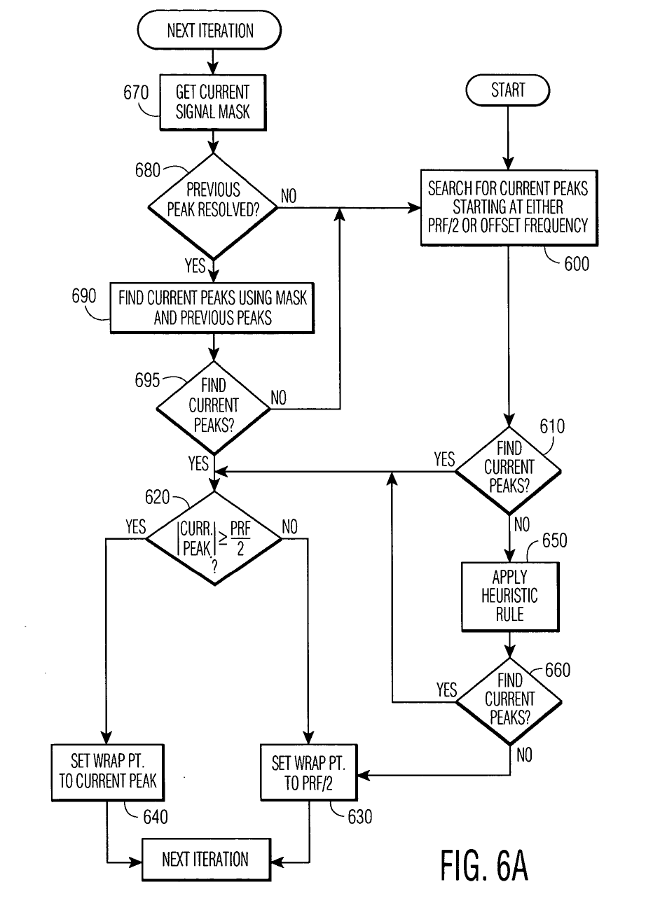

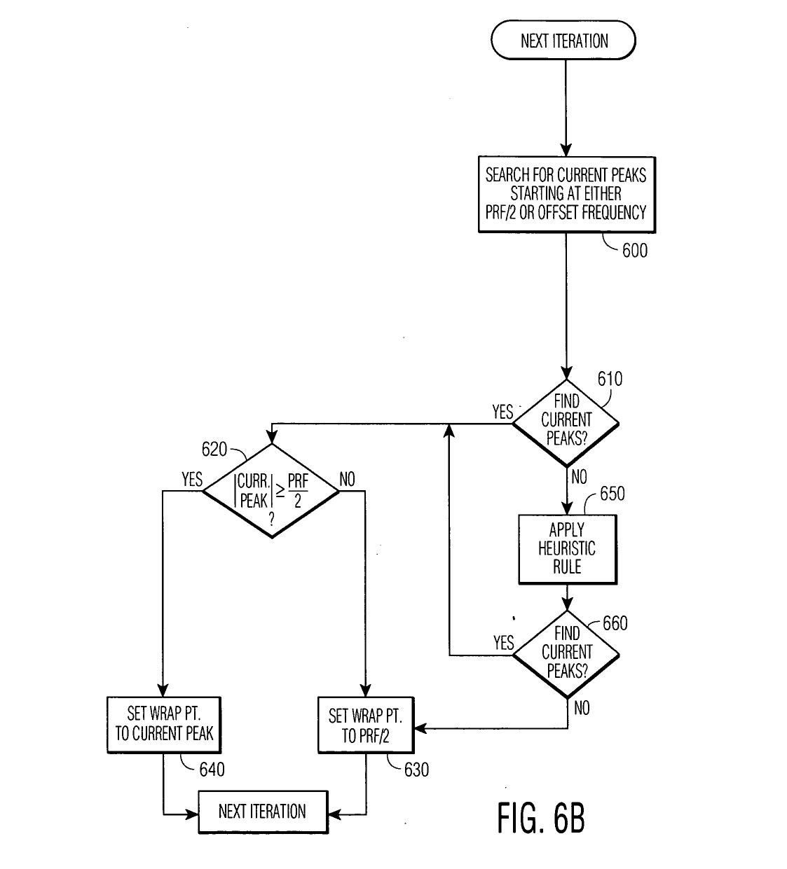

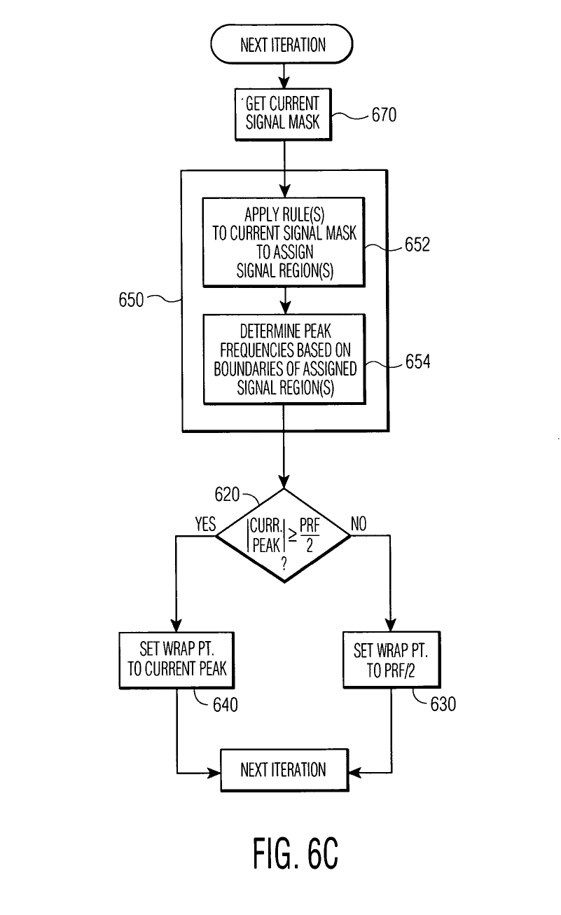

An exemplary flowchart of a method for avoiding aliasing effects according to one presently preferred embodiment of the present invention is shown in FIG. 6A. At the beginning of processing (START), a search for current peak frequencies is performed starting at either PRF/2 or at the baseline-offset wrap frequency of the spectral video display (Step 600).

If the search is successful (Step 610), it is determined whether the found peak frequencies are less than PRF/2 (Step 620). If they are less than PRF/2, the audio wrap frequency is set at +/−PRF/2 (Step 630). If one of the peak frequencies is greater than PRF/2, the audio wrap frequency is set to that peak frequency, or close to it (Step 640). After setting the audio wrap frequency in either Step 630 or Step 640, the method moves on to the next iteration, i.e., performs the same function for the next spectral line or column.

If the search in Step 600 is unsuccessful (Step 610), the system applies a heuristic rule, as discussed in above, in order to determine the peak frequencies (Step 650). If the frequencies are found in Step 650 (Step 660), it is determined whether the found peak frequencies are less than PRF/2 (Step 620), and, depending on the results of Step 620, the audio wrap frequency is set in either step 630 or 640. If the frequencies are not found in Step 650 (Step 660), the audio wrap frequency is set at +/−PRF/2 (Step 630). After setting the audio wrap frequency in either Step 630 or Step 640, the method proceeds to the next iteration.

In the next iteration, the current signal mask is obtained (Step 670), and it is determined whether the peak frequencies were resolved in the last iteration (Step 680). If the previous peak frequencies were not resolved (Step 680), the method returns to Step 600 in order to find the current peak frequencies. If the previous peak frequencies were resolved (Step 680), a search for the current peak frequencies is performed using the current signal mask, starting from the previous peak frequencies (Step 690). Step 690 is the “tracking” step, in which, by ‘tracking’ from the previous peak frequencies, the method quickly and efficiently finds the current peak frequencies. If Step 690 successfully finds the current peak frequencies (Step 695), the method continues in Step 620. If Step 690 does not find the current peak frequencies (Step 695), the method returns to Step 600.

The method of the flowchart in FIG. 6A is exemplary, and should not be considered to limit the scope of the invention in any way. One or more steps may be performed in a different order than described above, or may be omitted entirely, or may be changed, or may be replaced with a substitute step, or may be combined together to form one step, or may be divided into sub-steps, or any combination of these actions, without departing from the spirit of the invention. For example, more than one heuristic rule may be applied in step 650, or step 650 may be removed altogether. As another example, a step of creating a new signal mask could replace step 630, or could be added in case of repeated unsuccessful attempts at resolving the peak frequencies. As a further example, when implementing step 630 of FIG. 6A, one may wish to first determine if the wrap frequency is already set to +/−PRF/2, in which case re-setting the wrap frequency is unnecessary and won't be performed.

In another presently preferred embodiment of the present invention, the spectral column signal mask is used only to determine the present instantaneous peak frequencies, i.e., the peak frequencies are not tracked from one spectral column to the next. Thus, in this presently preferred embodiment, the search for the new peak frequency on the new spectral column signal mask starts at the same location each time. Although this embodiment starts at either PRF/2 or the offset frequency of the spectral display, any initial starting point may be used and, in some embodiments, it is contemplated that the starting point would be chosen at random or as the result of the application of an algorithm.

An exemplary flowchart of a method for avoiding aliasing effects according to another presently preferred embodiment of the present invention is shown in FIG. 6B. At the beginning of processing (or the beginning of the NEXT ITERATION), a search for current peak frequencies is performed starting at either PRF/2 or at the baseline-offset wrap frequency of the spectral video display (Step 600).

If the search is successful (Step 610), it is determined whether the found peak frequencies are less than PRF/2 (Step 620). If they are less than PRF/2, the audio wrap frequency is set at +/−PRF/2 (Step 630). If one of the peak frequencies is greater than PRF/2, the audio wrap frequency is set to that peak frequency, or close to it (Step 640). After setting the audio wrap frequency in either Step 630 or Step 640, the method moves on to the next iteration, i.e., performs the same function for the next spectral line or column.

If the search in Step 600 is unsuccessful (Step 610), the system applies a heuristic rule, as discussed in above, in order to determine the peak frequencies (Step 650). If the frequencies are found in Step 650 (Step 660), it is determined whether the found peak frequencies are less than PRF/2 (Step 620), and, depending on the results of Step 620, the audio wrap frequency is set in either step 630 or 640. If the frequencies are not found in Step 650 (Step 660), the audio wrap frequency is set at +/−PRF/2 (Step 630). After setting the audio wrap frequency in either Step 630 or Step 640, the method proceeds to the next iteration.

In the next iteration, the method repeats. Because there is no reference to previous spectra in this embodiment, start up is less of an issue and decision errors do not persist in time.

The method of the flowchart in FIG. 6B is exemplary, and should not be considered to limit the scope of the invention in any way. One or more steps may be performed in a different order than described above, or may be omitted entirely, or may be changed, or may be replaced with a substitute step, or may be combined together to form one step, or may be divided into sub-steps, or any combination of these actions, without departing from the spirit of the invention. For example, step 650 could be removed so that the next step after 610 (if the current peak frequencies are not resolved) would be setting the audio wrap frequency to PRF/2 in step 630. As another example, a step of creating a new signal mask could replace step 630, or could be added in case of repeated unsuccessful attempts at resolving the peak frequencies. As a further example, when implementing step 630 of FIG. 6B, one may wish to first determine if the audio wrap frequency is already set to +/−PRF/2, in which case re-setting it is unnecessary and won't be performed.

In yet another presently preferred embodiment of the present invention, a rule-based interpretation of the current signal mask is performed in order to assign each signal region in the signal mask to either the positive or negative side of the spectrum. Once each signal region is appropriately assigned, the peak frequencies can be easily found as the boundaries of the signal regions. The audio wrap frequency is set according to the determined position of the peak frequency. An exemplary flowchart of a method for avoiding aliasing effects according to this yet another presently preferred embodiment of the present invention is shown in FIG. 6C. At the beginning of processing (or the beginning of the NEXT ITERATION), the current signal mask is obtained in Step 670.

Once the current signal mask is obtained (Step 670), each signal region in the current signal mask is assigned to either the negative or positive side of the spectrum in Step 652. One or more rules may be used in order to implement Step 652, and these rules may be used either in the alternative, or progressively from less elaborate to more elaborate rules (i.e., proceeding to the next rule once it has been shown that the previous rule was unable to resolve the signal regions). Examples of such rules include: assigning any signal region which approaches within a certain distance of the edge of the clutter filter stopband to the side of the spectrum having that edge; and/or assigning the signal region to the side of the spectrum that it covers to the greater extent, when averaged over time. Furthermore, these rules may store and use the results of previous iterations in order to perform the assigning step.

Once the one or more signal regions are assigned (Step 652), the peak frequencies are determined based on the boundaries of the assigned signal regions in Step 654. Having resolved the peak frequencies (Step 654), it is determined whether the resolved peak frequencies are less than PRF/2 in Step 620, and, depending on the results of Step 620, the audio wrap frequency is set in either step 630 or 640. After setting the audio wrap frequencies in either Step 630 or Step 640, the method proceeds to the next iteration. i.e., performs the same function for the next spectral line or column.

The method of the flowchart in FIG. 6C is exemplary, and should not be considered to limit the scope of the invention in any way. One or more steps may be performed in a different order than described above, or may be omitted entirely, or may be changed, or may be replaced with a substitute step, or may be combined together to form one step, or may be divided into sub-steps, or any combination of these actions, without departing from the spirit of the invention. For example, if step 652 was unsuccessful (if the current signal regions can not be resolved), the method could proceed directly to Step 630 to set the audio wrap frequency to PRF/2.

The APPENDIX contains an exemplary implementation of a pseudo-program for setting the audio wrap frequency according to the presently preferred method shown in FIG. 6C. Steps 2 and 3(a) in the APPENDIX are an implementation of method steps similar to Steps 652 and 654 in FIG. 6C. Step 3(b) in the APPENDIX is a programming implementation of method steps similar to Steps 620-640 of FIGS. 6A-6C. The pseudo-program in the APPENDIX is intended as an example, and should not be considered to limit the scope of the present invention in any way. There are a multitudinous variety of ways to implement the present invention, whether in hardware, software, or firmware, as would be known to one skilled in the relevant art.

In order to implement any of the above-described processing, the received data signal may be transformed to and from the frequency domain (typically using FFT), or may be entirely processed in the time domain. Offsetting the audio wrap frequency from PRF/2(that is, the audio baseline shift) may be accomplished in either frequency domain or time domain. In the frequency domain, zeroes are inserted in the data array at the desired wrap frequency, making the data array larger by typically a power of two. Alternatively, in the time domain, a complex mixer, a filter, and a conjugate complex mixer, operating at a sample rate of 2×PRF or greater, can offset the wrap frequency from PRF/2. See either the Koo patent or the Otterson patent for examples of these techniques. Although these techniques are presented (and/or incorporated) herein, the present invention is not limited to these examples, and any technique capable of producing the desired result is within the scope of the present invention. Furthermore, the operations described herein may be performed by any combination of hardware, software, or firmware.

One benefit of the present invention, among others, is that the audio wrap frequency is automatically and dynamically adjusted based on the current received signals, thus guaranteeing an efficient and responsive allocation of PRF bandwidth over a 2×PRF range. By contrast, in prior art systems, such as the Koo and Otterson systems, PRF bandwidth allocation is user-adjusted (but otherwise static) and is based on the user's impressions, rather than on current received signals.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for ameliorating aliasing effects on audio output from an ultrasound imaging system performing Doppler spectral imaging comprising the steps of:
determining at least one of a current positive and a current negative instantaneous peak frequency of a spectral column in a Doppler spectral image using a signal mask;
setting a current audio wrap frequency at PRF/2 if the absolute value of the determined current peak frequency is below PRF/2; and
setting a current audio wrap frequency equal to, or greater than, the determined current peak frequency if the absolute value of the determined current peak frequency is greater than PRF/2.

2. The method of claim 1, wherein the step of determining at least one of a current positive and a current negative instantaneous peak frequency of a spectral column in a Doppler spectral image using a signal mask comprises the step of:
assigning each signal region in the current signal mask to either the positive or the negative side of the spectrum; and
determining at least one of a current positive and a current negative instantaneous peak frequency by determining the boundaries of each assigned signal region.

3. The method of claim 2, wherein the step of assigning each signal region in the current signal mask to either the positive or the negative side of the spectrum comprises the step of:
applying a heuristic rule in order to resolve signal regions in the current signal mask.

4. The method of claim 3, wherein the heuristic rule comprises the step of:
assigning a signal region to a side of the spectrum when said signal region approaches a clutter filter stopband on said side.

5. The method of claim 3, wherein the heuristic rule comprises the step of:
assigning a signal region to a side of the spectrum when said signal region covers said side to the greater extent, when averaged over time.

6. The method of claim 1, wherein the step of determining at least one of a current positive and a current negative instantaneous peak frequency of a spectral column in a Doppler spectral image using a signal mask comprises the step of:
searching for the at least one current peak frequency by starting at either PRF/2 or a baseline-offset wrap frequency of the spectral video display.

7. The method of claim 1, wherein the step of determining at least one of a current positive and a current negative instantaneous peak frequency of a spectral column in a Doppler spectral image using a signal mask comprises the steps of:
tracking at least one of a positive and a negative peak frequency of the Doppler spectral image using the signal mask from which an instantaneous peak frequency is derived; and
determining the at least one current peak frequency using at least the at least one tracked peak frequency.

8. The method of claim 7, wherein the step of determining at least one of a current positive and a current negative instantaneous peak frequency of a spectral column in a Doppler spectral image using a signal mask comprises the steps of:
taking as a starting point on the current signal mask a previous peak frequency; and
proceeding out from the starting point depending on whether the signal mask indicates the presence or absence of signal.

9. The method of claim 7, wherein, either at the beginning of processing or when a previous peak frequency is unresolved, the method further comprises the step of:
searching for the at least one current peak frequency by starting at either PRF/2 or a baseline-offset wrap frequency of the spectral video display.

10. The method of claim 9, wherein, if the step of searching is unsuccessful, the method further comprises the step of:
applying a heuristic rule in order to determine the at least one current peak frequency by resolving signal regions.

11. An ultrasound imaging system capable of ameliorating aliasing effects on audio output while performing Doppler spectral imaging comprising:
means for determining at least one of a current positive and a current negative instantaneous peak frequency of a spectral column in a Doppler spectral image using a signal mask;
means for setting a current audio wrap frequency at PRF/2 if the absolute value of the determined current peak frequency is below PRF/2; and
means for setting a current audio wrap frequency equal to, or greater than, the determined current peak frequency if the absolute value of the determined current peak frequency is greater than PRF/2.

12. The system of claim 11, wherein the means for determining at least one of a current positive and a current negative instantaneous peak frequency of a spectral column in a Doppler spectral image using a signal mask comprises:
means for assigning each signal region in the current signal mask to either the positive or the negative side of the spectrum; and
means for determining at least one of a current positive and a current negative instantaneous peak frequency by determining the boundaries of each assigned signal region.

13. The system of claim 12, wherein the means for assigning each signal region in the current signal mask to either the positive or the negative side of the spectrum comprises:
means for applying a heuristic rule in order to resolve signal regions in the current signal mask.

14. The system of claim 13, wherein the means for applying the heuristic rule comprises at least one of:
means for assigning a signal region to a side of the spectrum when said signal region approaches a clutter filter stopband on said side; and
means for assigning a signal region to a side of the spectrum when said signal region covers said side to the greater extent, when averaged over time.

15. The system of claim 11, wherein the means for determining at least one of a current positive and a current negative instantaneous peak frequency of a spectral column in a Doppler spectral image using a signal mask comprises:
means for tracking at least one of a positive and a negative peak frequency of the Doppler spectral image using the signal mask from which an instantaneous peak frequency is derived; and
means for determining the at least one current peak frequency using at least the at least one tracked peak frequency.

16. The system of claim 15, wherein the means for determining at least one of a current positive and a current negative instantaneous peak frequency of a spectral column in a Doppler spectral image using a signal mask comprises:
means for taking as a starting point on the current signal mask a previous peak frequency; and
means for proceeding out from the starting point depending on whether the signal mask indicates the presence or absence of signal.

17. A program of instructions for ameliorating aliasing effects on audio output from an ultrasound imaging system performing Doppler spectral imaging, wherein said program of instructions is stored on a computer-readable medium and is capable of being performed by one or more processors, said program of instructions comprising:
instructions for determining at least one of a current positive and a current negative instantaneous peak frequency of a spectral column in a Doppler spectral image using a signal mask;
instructions for setting a current audio wrap frequency at PRF/2 if the absolute value of the determined current peak frequency is below PRF/2; and
instructions for setting a current audio wrap frequency equal to, or greater than, the determined current peak frequency if the absolute value of the determined current peak frequency is greater than PRF/2.

18. The program of claim 17, wherein the instructions for determining at least one of a current positive and a current negative instantaneous peak frequency of a spectral column in a Doppler spectral image using a signal mask comprises:
instructions for assigning each signal region in the current signal mask to either the positive or the negative side of the spectrum; and
instructions for determining at least one of a current positive and a current negative instantaneous peak frequency by determining the boundaries of each assigned signal region.

19. The program of claim 18, wherein the instructions for assigning each signal region in the current signal mask to either the positive or the negative side of the spectrum comprises:
instructions for applying a heuristic rule in order to resolve signal regions in the current signal mask.

20. The program of claim 17, wherein the instructions for determining at least one of a current positive and a current negative instantaneous peak frequency of a spectral column in a Doppler spectral image using a signal mask comprises:
instructions for tracking at least one of a positive and a negative peak frequency of the Doppler spectral image using the signal mask from which an instantaneous peak frequency is derived; and
instructions for determining the current peak frequencies using at least the at least one tracked peak frequency.
